(19) United States
(12) Patent Application Publication
(10) Pub. No.: US 2022/0338883 A1
(43) Pub. Date: Oct. 27, 2022

(54) SURGICAL INSTRUMENT HANDPIECE

(71) Applicant: Aesculap AG, Tuttlingen (DE)

(72) Inventors: Roland-Alois Hoegerle, Tuttlingen (DE); André Buerk, Villingen-Schwenningen (DE)

(21) Appl. No.: 17/772,071

(22) PCT Filed: Oct. 29, 2020
(86) PCT No.: PCT/EP2020/080346

§ 371 Date: Apr. 26, 2022

(30) Foreign Application Priority Data
Oct. 29, 2019 (DE) 10 2019 129 218.2

(51) Int. Cl. A61B 17/16; A61B 90/70

(52) U.S. Cl. CPC A61B 17/1622; A61B 90/70; A61B 17/1631; A61B 2090/0813

(57) ABSTRACT

A surgical instrument handpiece for a surgical instrument, a corresponding surgical instrument, a corresponding medical product set having the surgical instrument handpiece in combination with at least one accessory, a corresponding rinsing device, and a corresponding cleaning method for the internal flushing of the surgical instrument handpiece. The surgical instrument handpiece includes a handle section for proximal handling by an operator and a shaft section that extends from the handle section in a distal longitudinal direction. A tool is arranged or arrangeable by a user in a distal outlet opening of the shaft section at the distal end opposite the handle section. The shaft section has at least one narrowed distal tip section with a reduced cross-sectional area in the region of the distal outlet opening.

### CROSS-REFERENCES TO RELATED APPLICATIONS

This application is the United States national stage entry of International Application No. PCT/EP2020/080346, filed Oct. 29, 2020, and claims priority to German Application No. 10 2019 129 218.2, filed Oct. 29, 2019. The contents of International Application No. PCT/EP2020/080346 and German Application No. 10 2019 129 218.2 are incorporated by reference herein in their entireties.

### FIELD

The present disclosure relates to a surgical instrument handpiece as it is used for the reception of or for driving a surgical tool in the broadest sense, in particular a rotatable tool like a cutter, a drill, a grinding head or the like. Furthermore, the present disclosure relates to such a surgical instrument as well as to an corresponding medical product set having the surgical instrument handpiece in combination with at least one accessory. Moreover, there are proposed an corresponding rinsing device, like a cleaning and disinfection unit or a thermodisinfector, as well as an corresponding cleaning method for the internal flushing of the surgical instrument handpiece.

### BACKGROUND

It is known in the prior art of modern minimally invasive surgery, in particular of neurosurgery and spine surgery, to use surgical instrument handpieces for the purpose of working of for example bones, cartilages, vertebras, etc. DE 10 2013 111 194 A1 of the present applicant, for instance, which hereby is explicitly incorporated as part of the present patent application by reference thereto, refers to a surgical instrument handpiece according to the preamble of the claim. Here the respective surgical instrument handpiece usually comprises at least one receptacle or coupling or interconnection for a coupleable, preferably rotatably drivable, tool. Medical indications in which surgical instrument handpieces are used comprise arthroscopies and arthroscopic surgeries for the purpose of the examination and/or treatment of joints, orthopedic interventions or surgeries, spine surgery, maxillary surgery, neurosurgery, and so on.

During the use of the surgical instruments (or instrument handpieces), the distal end of the instrument (or of the instrument handpiece) comes into contact with organic and inorganic substances, as for instance body fluids, bone abrasion, which tend to accumulate or settle in form of deposits or attachments at and/or in the instrument (or instrument handpiece). Therefore, the usage of reusable surgical instruments (or instrument handpieces) requires a proper preparation or processing, in particular cleaning and/or disinfection, before and/or after each use thereof, in which preparation or processing the soilings adhering in and at the instrument are removed again for a repeated sterile use of the instrument.

Especially the cleaning of the lumens or hollow bodies of the instrument handpiece with its internal surfaces and/or parts is a particular challenge. To this end, the instrument handpiece or the hollow body of the instrument handpiece will be flushed with a cleaning fluid, in particular with a cleaning solution. The internal flushing with the cleaning fluid shall remove particles of dirt which have accumulated at the internal surfaces and/or parts of the hollow body or the instrument handpiece, and shall discharge them from the hollow body or the instrument handpiece.

In this process, in particular the cleaning of the intermediate spaces in the hollow body of the instrument handpiece, in particular of its distal ball bearings (facing the patient) for the reception of a rotatably drivable tool like a cutter or a drill, has proved to be difficult. Due to the low resistance, when flowing through the instrument handpiece the cleaning fluid flows almost exclusively through the inner rings or along the inner surfaces of the inner rings of the distal ball bearings so that the balls, cages and intermediate spaces of said ball bearings will only be subject to a lesser cleaning effect.

Said problem has already been addressed in prior art. For instance the patent application of the present applicant with the officially issued file number DE 10 2018 133 503.2 the disclosure of which is hereby explicitly incorporated as part of the present patent application by reference thereto, discloses a separate flushing device comprising or being composed of a shaft-type flushing insert for an internal cleaning of an instrument handpiece of a surgical instrument, in particular a flushing device for cleaning the distal ball bearings in the internal space of an instrument handpiece. In doing so, the flushing device proposed therein will be inserted into or attached in a tool receptacle or in a tool receptacle shaft of the instrument handpiece after the surgical use thereof for the purpose of cleaning.

Although the above presented solution from the prior art convinces with regard to the achievable cleaning efficiency, there is, however, still the disadvantage that, in addition to the instrument handpiece, together with the flushing device disclosed therein a separate device, namely the shaft-type flushing insert, has to be provided and has to be inserted into the instrument handpiece prior to the cleaning cycle in the Central Sterile Services Department (CSSD) or Central Sterilization or Reprocessing Unit for Medical Devices (RUMED) There is the risk that the separate flushing device can get lost. Furthermore, the handling and the application of the flushing device have to be described and implemented separately.

Furthermore, independent of the above discussed cleaning aspect there exist further disadvantages for the prior art concerning surgical instrument handpieces with a view to their medical, in particular surgical application. In fact, from the aspect of the outer dimensioning or the clear dimensions of the shaft section, these disadvantages ensue as negative. For example, with respect to minimally invasive, least possible traumatizing surgeries there exists a need for dimensions of the surgical instrument handpieces which are as small as possible or which are even smaller and smaller. On the one hand, for the visualization of the surgical operation or of the handling within the patient in real time, in particular via an endoscopic camera image with a reproduction on a surgery monitor for the surgeon, there exists the disadvantage in the prior art with regard to the instrument handpieces that they obstruct or shadow the view onto the tissue according to their dimensioning. In particular in the field of microsurgery, said obstruction of the view will turn out to be all the greater and all the more disadvantageous. On the other hand, with regard to the surgical access, for instance in the field of brain surgery, or with regard to the operable indications as such there exists a need for a smaller dimensioning of the surgical instrument handpiece or for a corresponding expansion of the surgical field of application.

US 2017/0 120 451 A1 discloses a surgical instrument handpiece in the form of an assembly for holding a tool, wherein the assembly may selectively reduce and/or eliminate vibrations received and felt by a user. Reducing vibrations may reduce or eliminate chatter at a working end of a tool. For this purpose, a vibration-dampening middle intermediate element is positioned between the one shaft section for the reception of the tool and a handle section of the surgical instrument handpiece. Here, in the transition area of the middle intermediate element there are disclosed two internal diameters being slightly different from each other, but in a section lying proximally from a ball bearing, and, thus, referring to the proximal shaft section within the handle section. Furthermore, in the prior art it has to be regarded as potentially problematic that the entire input pressure of the cleaning fluid (in the sense of a pressure difference or an excess pressure compared to the atmospheric ambient pressure), which is proximally provided in a rinsing device, like a cleaning and disinfection unit, from an external source to a number of several connected instrument handpieces which have to be cleaned, will be distributed proportionally according to the number into a respective rinsing pressure per each instrument handpiece and will, thus, be reduced. As a consequence, in case of a (too) high loading or charging of the rinsing device in the prior art, the unsatisfactory case may occur that the respective rinsing pressure will fall below a minimum value required for the purpose of a reliable and sufficient fluid cleaning. Said technical disadvantage of a rinsing pressure which is possibly too low, respectively, can arise in particular in a situation with a high hospital capacity utilization.

Furthermore, in the prior art there can exist the unfavorable situation that a free flow cross-section (or an inner void volume) of the instrument handpiece being available for the through-flow with the cleaning fluid is widened along the longitudinal direction or flow direction from proximal to distal. As a consequence, the mechanical cleaning effect from proximal to distal will deteriorate, in addition to the general flow pressure losses, in particular because of the pipe friction and other flow resistance coefficients.

### SUMMARY

Hence, the invention is based on the object to create a surgical instrument handpiece for a surgical instrument which overcomes the above explained disadvantages of the prior art. First of all, an even safer instrument handpiece which can be cleaned or sterilized by means of an internal flushing shall be (alternatively) facilitated. In particular, the constructional design of the instrument handpiece shall enable a targeted and powerful cleaning of the distal ball bearings. Moreover, an additional object is to supply the user with a further field of application of operable indications. In addition, one further object is to simplify the processes at the Central Sterile Services Department (CSSD) so that said processes will become more cost-effective and less prone to errors.

The surgical instrument handpiece for a surgical instrument as a first aspect of the present disclosure comprises a handle section used for the proximal handling by an operator, and a shaft section which extends from the handle section in a distal longitudinal direction. Therein, a tool is arranged or arrangeable by a user, like the operator or an operating room staff, in a distal outlet opening of the shaft section at the distal end opposite the handle section. By a tool in the sense of the disclosure there is meant any device or unit by means of which an operator can treat and/or work the body or body parts of a patient or implants or the like, wherein the tool is guided by the operator by means of the instrument handpiece.

According to the disclosure, the shaft section has at least one narrowed distal tip section in the region of the distal outlet opening.

Here the term “narrowed” or “narrowing” refers to a cross-sectional area at the distal end, in particular to a clear external dimension of the shaft section, preferably furthermore also to an inner flow cross-section.

Thereby, in the sense of the present disclosure the term “distal” means the application-technical perspective of the operator or user handling the instrument handpiece according to the disclosure, which corresponds to the side facing the patient. As a consequence, here the term “proximal” designates the side facing the operator or user, i.e. the side facing away from the patient.

Consequently, according to the disclosure the shaft section is subdivided into at least two (longitudinal) sections along its longitudinal direction, i.e. into a first section and a second section having different cross-sectional areas. Here the comprised first section with a smaller first cross-sectional area is referred to as a narrowed distal tip section of the (entire) shaft section. And the comprised second section with a second cross-sectional area being larger when compared to the first section is designated as a non-narrowed section of the (entire) shaft section. In other words, in a distal longitudinal direction the narrowed first section/the distal tip section adjoins the shaft section which extends in the distal longitudinal direction from the handle section/adjoins the non-narrowed second section of the (entire) shaft section. That is to say, a first length along which the first section/the distal tip section extends in the form of a narrowing corresponds to a part of the entire length of the (entire) shaft section that is designated as a second length. Consequently, the difference from the (entire) second length and the first length refers to the (remaining) second section or the non-narrowed region of the shaft section.

As a result, according to the disclosure the flow velocity of the cleaning fluid and, thus, the hydrodynamic cleaning effect will be increased due to the narrowing of the distal flow cross-section.

Due to the constructional design of the instrument handpiece according to the disclosure, in case of an internal flushing a cleaning fluid will be specifically guided in a flow direction from proximal to distal up to the distal outlet opening, and, hence, will no longer mostly flow through the comparatively large and, consequently, only a very low flow resistance offering opening of the prior art of an inner ring of a distal ball bearing in a manner which is less effective or which is ineffective. In other words, the present disclosure serves to reduce the proportion of a rinsing flow of the cleaning fluid which is not effectively guided or is even misguided. This will be caused while vastly wide-ranging maintaining flow pressure being sufficient, ideally excessively high, for the cleaning effect along the longitudinal axis of the instrument handpiece as a main flow direction.

Furthermore, the optimized outer contour of the shaft section that is narrowed according to the disclosure in the distal tip section improves in an advantageous manner the view access for the operator during a surgery or intervention.

In addition, according to the disclosure simplified processes in the Central Sterile Services Department (CSSD) will be supported which has a positive effect on reduced operating costs, on the one hand, and on the quality assurance or reliability on the other hand. The new design according to the disclosure of the distal tip/of the tip section comprised by the shaft section, of the instrument handpiece increases the cleaning effect, in particular with regard to the distal ball bearings and/or with regard to an inner surface section of the (distal) tip section during the manual or mechanical cleaning, without having to insert additional products like a special rinsing device.

It is irrelevant in the sense of the disclosure whether, in addition to the at least one narrowed distal tip section, also further sections of a further different cross-sectional area are comprised by the shaft section, in particular by the first section/the distal tip section itself. In order words, it is conceivable that further peripheral shaft shoulders and/or shaft steps are formed.

Furthermore, in the sense of the disclosure it is irrelevant in which kind a connection, preferably arranged at the proximal end of the handle section, or an interface of the instrument handpiece to the external power supply or for the handling or driving of the, preferably rotatably drivable, tool is designed. Depending on the purpose of use and the intended tool speed, a hydraulic, pneumatic and/or electromotive drive can be provided or can be operatively connected.

Moreover, in the sense of the disclosure it is irrelevant that the narrowing or, respectively, the reduced/narrowed cross-sectional area has a specific form. Consequently, herein said terms are not to be interpreted in a manner restricted to only longitudinal forms with a constant round cross-section such that radially peripheral shaft shoulders and/or radial steps are formed. Instead, the present terminology also comprises any narrowings with a cross-section being variable along their longitudinal axis and/or with a cross-section having an uneven or non-round form, as for instance an oval, rectangular, convex and/or concave form. In particular, the narrowing may only be formed in an angular segment or it may form an asymmetrically reduced cross-sectional area.

Very often, however, radially constant or round cross-sectional areas or narrowings of the shaft section may be preferred due to production-technical reasons as well as also under application-technical aspects, in particular flow-dynamic aspects.

Consequently, under the last mentioned aspects it is preferred that the surgical instrument handpiece is further improved such that a first diameter of the narrowed distal tip section is smaller by a diameter relation factor of a maximum of 95 percent, preferably of a maximum of 85 percent, more preferably of approximately 79 percent in comparison to a second diameter of a non-narrowed region of the shaft section.

Alternatively or cumulatively, the first diameter preferably amounts to between 3.5 and 5.3 millimeter, preferably to between 4.0 and 5.0 millimeter, more preferably to between 4.3 and 4.5 millimeter.

Thereby there may in particular be preferably provided a change in diameter or a narrowing of the second diameter, like an outer diameter of the shaft section, of approximately 5.6 mm, in particular related to a region abutting at the handle section, down to approximately 4.4 mm for the first diameter, like an outer diameter of the narrowed distal tip section.

Preferably, the surgical instrument handpiece is further developed such that a first length of the distal tip section lies between 5 and 40 millimeter, preferably between 10 and 30 millimeter, more preferably between 18 and 22 millimeter.

Alternatively or cumulatively, thereby the first length, as far as it is related to or compared with or standardized with respect to an entire second length of the shaft section, preferably amounts to a percentage share of the length of at least 5 percent, preferably of at least 20 percent, more preferably of at least 35 percent.

In particular there may be preferred an instrument handpiece in which the distal tip section with a width of preferably approximately 4.4 mm extends to approximately 20 mm as a first length, while the second (entire) length is chosen or set according to an application-technically or surgical-operatively optimal length for the (entire) shaft section preferably having a width of approximately 5.6 mm.

On that point it could be determined experimentally by means of exemplary prototypes that such particularly preferred embodiments of the disclosure represent a further optimized balance of all application-technical dimensions or independent groups of technical problems or objectives. That is to say, the above mentioned surgical-operative aspects relate to a first group of technical tasks and the flow-dynamic effects in the sense of or for the purpose of the following cleaning relate to a second group of technical tasks. In this respect, the first group relates to a first period of application during the surgical or operative use, in particular by an operator as a first user; and the second group relates to a second period of application after the performed surgical or operative use, in particular by the staff in the field of activity of cleaning or sterilization of surgical instruments and tools as a second user.

It goes without saying, however, that the disclosure is not to be restricted to the above mentioned particularly preferred embodiments. Not least in the case of a miniaturization—as already known from the scientific basics of the parameter or quantity problem of flow dynamics—there are to be chosen or to be preferred deviating or other absolute and/or relative dimensions.

Preferably, a transition region designed as a shoulder from the narrowed distal tip section to the non-narrowed region of the shaft section is formed in a rounded shape and/or in a gradually tapering off shape and/or in a chamfered shape. Such an avoidance of an angular or abrupt transition between the first section and the second section offers the advantage of less dirt adhesion or of a reduced tissue traumatization during the operative handling or during the insertion into a tissue opened up by the surgeon.

Preferably, the surgical instrument handpiece is adapted to be inserted into a rinsing device, like a cleaning and disinfection unit, in such a manner that the rinsing pressure is optimized that is present at the distal outlet opening during an internal flushing of the instrument handpiece with a cleaning fluid, preferably with a hydrophilic or lipophilic cleaning solution, in the flow direction from proximal to distal. For this, the above mentioned rinsing pressure is larger than 10 mbar, further preferred larger than 90 mbar, even more preferred larger than 160 mbar and in particular larger than 500 mbar. Alternatively or cumulatively, the rinsing pressure, as far as it is related to or standardized with respect to a proximally provided input pressure of the cleaning fluid, will remain maintained in a proportion of at least 20 percent, preferably of at least 50 percent, further preferred of at least 80 percent. In this manner, a particularly high cleaning effect can be achieved, as is verifiable in particular by means of standardized cleaning test values as they are used in the complex technical knowledge and the legal guidelines for the preparation of medical devices.

This is particularly advantageous in order to effectively address the problem arising in the prior art of a rinsing pressure or flow pressure which is reduced in part considerably along the longitudinal direction of the instrument handpiece from proximal to distal/along the flow direction (or along the direction of the flow lines). Said problem in conventional instrument handpieces can be of a particular severity if or in so far as the (inner) flow cross-sectional area will not only not be constant but will even increase, in particular even significantly increase, along the longitudinal direction of the instrument handpiece from proximal to distal/along the flow direction. The continuity equation for the (incompressible) flow says A·v=V′=const.; wherein A stands for the (inner) cross-sectional area of the flow; v stands for a (an averaged) flow rate of the cleaning fluid; and V′ stands for a (an inner) volume flow of the cleaning fluid. Thus, according to the continuity equation for the (incompressible) flow this means that the flow rate (correlating negatively to the flow cross-sectional area) will decrease correspondingly. As a consequence, the flow term for the kinetic energy will decrease (see Bernoulli's equation), whereby in the prior art also a mechanical cleaning effect decreases in an unfavorable manner at the same time.

Preferably, a Reynolds number/Re for the through-flow in the narrowed distal tip section can mark a turbulent region, in particular above Re=2300. Thereby, as a density of the fluid and as a dynamic viscosity of the fluid the material values of the cleaning fluid, in particular of water, are to be included into the Reynolds number. Furthermore, the (averaged) flow rate of the cleaning fluid v is to be used as a flow velocity of the fluid. Thereby, preferably a diameter of a (flow) cross-section being available to the flow, more preferably a diameter of the inner surface section of the (distal) tip section, is defined as a characteristic length of the body (or of the pipe), also referred to as reference length. A turbulent region can bring about advantages in terms of a particularly powerful cleaning in the case of most obstinate soilings or contaminations.

As an alternative to a Reynolds number marking a turbulent region in the narrowed distal tip section, it may be particularly preferred that the Reynolds number marks a laminar region in the narrowed distal tip section. Particularly preferred, Re may lie between 1000 and 2000. This has the advantage of a uniform through-flow through the instrument handpiece according to the disclosure while avoiding any pulsation and/or any interactions between the fluid and the walls. This enables a particularly uniform, low-noise and low-vibration cleaning operation of a rinsing device.

Preferably, the shaft section comprises an inner roller bearing arrangement for a rotatable mounting of the arranged or arrangeable tool, preferably at least in part in the region of the narrowed distal tip section. Thereby, the roller bearing arrangement comprises at least one distal roller bearing and at least one proximal roller bearing. Furthermore, thereby the at least one distal roller bearing and the at least one proximal roller bearing are arranged in a manner spaced apart from each other by means of a bearing cage that is continuously formed between them.

As a result of this, according to the disclosure the unfavorable flow behavior as listed in the prior art is avoided in advance, which flow behavior includes a maximum flow of the through-flow along a cylinder axis of the shaft section or in the center of the second cross-sectional area. Thereby, said disadvantageous flow behavior in the prior art results from the hydrodynamic law of the path according to the least flow resistance and/or according to the flow condition known as the wall adhesion condition, in particular in the case of a pipe flow, and/or even further with respect to an inside of a roller bearing arrangement according to the through-flow through a bed (“Pre-Darcy”). In other words, it is a result of the prior art that there is a disadvantageous hydrodynamic distribution of the flow rate (or of the vector portion in the longitudinal direction of the instrument handpiece from proximal to distal) with a maximum in the center of the shaft section or along an axis of rotation of the instrument handpiece.

In particular, an entire length of the continuously formed bearing cage may amount to at least 90% of the first length of the distal tip section. Alternatively or cumulatively, a spacing length between the at least one proximal roller bearing, for instance (but not in a limiting manner) of a center line thereof, and the at least one distal roller bearing, for instance (but not in a limiting manner) of a center line thereof, and corresponding to the continuously formed bearing cage may amount to at least 60%, further preferred to at least 70% and particularly to at least 78% of the first length.

The particularly preferred embodiment of the present disclosure which refers to a continuously formed bearing cage overcomes the above described disadvantage of the prior art in a particularly effective manner. In this respect, a forced through-flow through the inner roller bearing arrangement and/or along the inner surface section is effected. Hence, the mechanical cleaning effect by means of the cleaning fluid will be further intensified in the state connected to the rinsing device, in particular to the cleaning and disinfection unit. As a result of this, a reliable and full fluid cleaning exactly at the points or surfaces where soilings can adhere takes place, and this even in a quite effective manner. In other words, the efficiency of the fluid cleaning/the flushing is increased by avoiding that the cleaning fluid will take the path of the least (flow) resistance and will escape centrally. Instead, according to the disclosure the cleaning fluid is specifically guided in the direction of the surfaces to be cleaned, where it shall develop the mechanical cleaning effect, namely in particular within the at least one distal roller bearing and the at least one proximal roller bearing and/or along the inner surface section of the narrowed distal tip section.

In the present case, as roller bearing(s) (arrangements) there are to be regarded such bearings in which, in contrast to the lubrication within slide bearings or friction bearings, rolling elements like for instance balls, cylinders, needles, barrels or cones reduce the frictional resistance between an inner ring and an outer ring.

In the embodiment with the inner roller bearing arrangement, preferably with the continuously formed bearing cage, in addition a specific, i.e. targeted flow control or a forced through-flow of the cleaning fluid through a gap void volume which arises or is formed between an inner surface section of the (distal) tip section and an outer surface section of the bearing cage is effected.

Furthermore, in contrast to the prior art, preferably a situation may be preferred in which (almost) all other possible flow paths (with the exception of the gap void volume) of the instrument handpiece, like an inner bore through at least one complementarily configured component like a drive shaft, are closed. In other words, the forced through-flow takes place without exception (or completely) through the gap void volume.

In particular, the gap void volume may refer to a ring gap void volume. In this respect it is further preferred that the inner surface section of the (distal) tip section and the outer surface section of the bearing cage may be designed in a cylindrical shape, more preferred they may be arranged concentrically, i.e. on a coinciding central axis. Thereby it is irrelevant for the present disclosure whether or to what extent there are present in particular manufacturing tolerances or fits. In particular, the latter may alternatively or cumulatively be configured to take into account a design/construction criterion and/or an operational criterion, preferably a concentricity characteristic of the inner roller bearing arrangement and/or a machine-dynamic parameter.

A corresponding (respective flow) cross-sectional area corresponds to the (respective) (ring) gap void volume. Thus, a cylindrical ring gap cross-sectional area is formed between an outer ring gap outer diameter (of a cylindrical inner component, in particular of the continuously formed bearing cage) and an inner ring gap inner diameter for the forced through-flow. Consequently, the ring gap cross-sectional area is calculated as a (respective flow) cross-sectional area based on the subtraction of the two circular areas with the ring gap outer diameter or, respectively, with the ring gap inner diameter.

It is particularly preferred that the (respective flow) cross-sectional area, in particular the cross-sectional area of the ring gap, cannot increase in the flow direction of the cleaning fluid or along the second length of the entire shaft section (from proximal to distal), in particular from a proximal region in the transition to the handle section up to a distal region at the proximal roller bearing and/or up to the outlet cross-sectional area. In other words, in particular the ring gap cross-sectional area may remain constant and/or may be reduced in the flow direction. Thereby it is further preferred that the narrowing proceeds continuously/does not show any discontinuities. This helps to avoid any abrupt change of the flow condition. In this manner, in particular local dead zones or turbulences or throttling effects which can entail local decreases of the mechanical fluid cleaning are avoided.

In the preferred embodiment with the inner roller bearing arrangement as the continuously formed bearing cage, the bearing cage ring gap cross-sectional area formed around the outer circumference of the (central) bearing cage is obtained from the subtraction of the two circular areas with the diameter of the inner surface section as the ring gap outer diameter or, respectively, with the bearing cage outer diameter as the ring gap inner diameter.

Preferably, the bearing cage ring gap cross-sectional area in the narrowed distal tip section may amount to less than or equal to 3.5 mm², further preferred to less than or equal to approximately 3 mm² and in particular preferred to less than or equal to 2.8 mm².

Alternatively or cumulatively, the bearing cage ring gap cross-sectional area in the narrowed distal tip section may be less than or equal to a flow cross-sectional area through which a through-flow is possible in a region being proximal thereto, in particular it may be less than or equal to a proximal shaft section ring gap cross-sectional area. Thereby, the proximal shaft section ring gap cross-sectional area may refer to the region in the transition to the handle section, wherein said region is proximal to the shaft section, and/or to a region of the tool receptacle in the shaft section, wherein said region is proximal to the proximal roller bearing.

Thereby, in particular the (distal) bearing cage ring gap cross-sectional area may amount to less than or equal to 85%, further preferred to less than or equal to 80% and in particular preferred to less than or equal to 76.5% of the proximal shaft section ring gap cross-sectional area.

Thereby, alternatively or cumulatively in particular the (distal) bearing cage ring gap cross-sectional area may amount to less than or equal to 150%, further preferred to less than or equal to 130% and particularly preferred to less than or equal to approximately 122% of a (proximal) instrument handpiece input cross-sectional area. Thereby, the instrument handpiece input cross-sectional area refers to a flow cross-sectional area of the instrument handpiece that is arranged in the connection area to the rinsing device and permits a (free) through-flow. In particular the instrument handpiece input cross-sectional area indicates a minimum of the flow cross-sectional area/a bottleneck when it is referred to the through-flow through the entire instrument handpiece.

It is particularly preferred that the flow quantity/the volume flow of the cleaning fluid and/or the flow rate of the cleaning fluid through the at least one distal roller bearing and/or through the at least one proximal roller bearing is/are increased. In particular a longitudinal vector portion of the flow rate in the longitudinal direction of the shaft section or in the longitudinal direction of the tip section is increased. This results in the advantage that zones with a flow rate of the cleaning fluid being only inadequately high with respect to the mechanical cleaning effect, and/or dead zones are avoided.

The increase of the flow rate, in particular of the longitudinal vector portion, through the inner roller bearing arrangement or along the inner roller bearing arrangement can be determined or quantified in particular according to a respectively corresponding bearing cage intensification factor.

On the one hand, the respectively corresponding bearing cage intensification factor, as regards a primary or first bearing cage intensification factor, can be determined by comparing or relating a situation according to the disclosure, i.e. with a narrowed distal tip section, with or to or in relation to a usual situation according to the prior art as specified at the outset, i.e. without the presence of a narrowed distal tip section.

Preferably, the first bearing cage intensification factor, in particular related to the distal roller bearing, may amount to greater than or equal to 1.5, further preferred to greater than or equal to 2.5 and particularly preferred to greater than or equal to 3. Insofar as the flow rate to a quadratic exponent goes down into the calculation of a term of the kinetic energy of a flow, an increase of the first bearing cage intensification factor effects a noticeable increase of the kinetic energy and, thus, of the mechanical cleaning performance. In particular it is effected in a positive manner that all zones or all surfaces to be cleaned will be reliably flushed and will be powerfully cleaned by the fluid.

On the other hand, the respectively corresponding bearing cage intensification factor, as regards a secondary or second bearing cage intensification factor, can be determined by a comparison with or in relation to a situation according to the disclosure, i.e. with a narrowed distal tip section. Accordingly, the second bearing cage intensification factor indicates for the further preferred embodiment with the continuously formed bearing cage how said factor still further improves the situation according to the disclosure with a narrowed distal tip section (without the continuously formed bearing cage). Preferably, the second bearing cage intensification factor, in particular in relation to the distal roller bearing, may amount to greater than or equal to 1.1, further preferred to greater than or equal to 1.5, and particularly preferred to greater than or equal to 2.

In the preferred embodiment with the inner roller bearing arrangement in the manner of the continuously formed bearing cage a width of the bearing cage ring gap is defined as the reference length for the Reynolds number. Here, the width of the bearing cage ring gap is calculated based on the subtraction of the outer diameter of the bearing cage as the ring gap inner diameter from the diameter of the inner surface section as the ring gap outer diameter. Apart from that, reference is made to all details disclosed in the foregoing with regard to the Reynolds number. In the preferred embodiment it is of particular advantage that the Reynolds number is lowered. In particular, with respect to the through-flow an otherwise (starting) turbulent region can be reversed by the insertion of the bearing cage and, thus, by a corresponding reduction of the reference length in a laminar region.

On the whole, in the embodiment with the inner roller bearing arrangement, preferably with the continuously formed bearing cage, advantages will be achieved to the effect that the cleaning effect is even further improved during the internal flushing of the instrument handpiece due to the fact that a specific, i.e. targeted flow control of the cleaning fluid takes place. In particular, a forced through-flow or forced convection of the cleaning fluid through the at least one distal roller bearing and/or the at least one proximal roller bearing takes place, which ensures an intensive flow around the rolling elements. As a result thereof, an even further intensified hydromechanical effect is obtained when the continuously formed bearing cage is present. Thereby there can be obtained a reliable fluid cleaning even in such a fluid cleaning operation of the rinsing device, in particular the cleaning and disinfection unit, which is loaded with a high number of the connected instrument handpieces, i.e. at particularly many, in particular (almost) all input connections or adapters or flanges provided therein. That is to say, also in case of smaller partial volume flows into which a total volume flow of the cleaning flow being available to the rinsing device is divided or split according to the number of the connected instrument handpieces, a sufficient fluid through-flow and, consequently, an adequately strong mechanical cleaning effect is guaranteed.

As already explained above, the surgical instrument handpiece is adapted to be inserted into a rinsing device, like a cleaning and disinfection unit, in order to be able to perform an internal flushing of the instrument handpiece with a cleaning fluid, preferably with a hydrophilic or lipophilic cleaning solution, in the flow direction from proximal to distal. In the case of the preferred embodiment of the surgical instrument handpiece (in particular with a continuously formed bearing cage) directly described above, the rinsing pressure then being present at the distal outlet opening amounts preferably to larger than 600 mbar, preferably to larger than 700 mbar and further preferred to approximately 800 mbar. Hence, said particularly preferred embodiment guarantees to the highest degree an effective cleaning.

Preferably, the bearing cage is of a completely closed design. This has the advantage of a maximum cleaning power for the distal roller bearings. Alternatively, the bearing cage is permeable to a fluid in a small area-related hole volume fraction. Thereby, the hole volume fraction may amount preferably to less than 40 percent, further preferred to less than 15 percent, particularly preferred to less than 8 percent. This permits a further optimization in the sense of a cleaning effect as uniformly as possible on the basis of a flow guidance refined over and along the bearing cage. The flow guidance can for instance be achieved by means of a fluid-dynamic modeling or by means of calculation methods with the aid of finite volume elements of the inner flow space of the instrument handpiece.

Preferably, the at least one distal roller bearing and/or the at least one proximal roller bearing, preferably all roller bearings of the inner roller bearing arrangement, have non-spherical rolling elements. Thereby it is further preferred that the at least one distal roller bearing and/or the at least one proximal roller bearing is designed as cylindrical roller bearing and/or as needle bearings. The rolling elements of cylindrical roller bearings are circular cylinders. Cylindrical roller bearings are manufactured in different designs, as described in the DIN 5412 Standard the disclosure of which is hereby incorporated by reference thereto. A needle bearing has circular-cylindrical rolling elements which are called needles, wherein said needles have very large lengths in comparison to the rolling element diameter (relation factor greater than or equal to approximately 2.5). Needle bearings are standardized in the DIN 617 Standard the disclosure of which is hereby incorporated by reference thereto. In these preferred embodiments with non-spherical rolling elements, the roller bearing arrangement is characterized by a large radial load capacity as well as by a flat or compact constructional design. Furthermore, during the internal flushing with a cleaning fluid, in cylindrical roller bearings and/or needle bearings the tendency to a non-uniform or pulsating flow behavior is reduced when compared to spherical rolling elements or ball bearings, which is caused by the reduced gap channel width between the outer lateral surface of the bearing cage and the inner peripheral surface of the shaft section. As a result of this, also the cleaning effect is favorably increased.

Preferably, the at least one distal roller bearing and/or the at least one proximal roller bearing, preferably all roller bearings of the inner roller bearing arrangement, comprise ceramic rolling elements or ceramic needles. Thereby the mechanical strength, in particular the permanent load behavior, is improved which has the positive result of longer lifetimes and longer service intervals. Furthermore, the rolling elements may be made even smaller in their dimensions so that an even flatter designed rolling element construction can be implemented. Thereby, the gap channel width between the outer lateral surface of the bearing cage and the inner peripheral surface of the shaft section can be reduced even further. It is also conceivable that full-ceramic bearings are used. In particular, in addition to the rolling elements, also the bearing rings consist of ceramic materials.

According to a second aspect of the present disclosure there is proposed a surgical instrument having an instrument handpiece according to the disclosure and also a tool which preferably is rotatably driven and/or can be rotatably driven. Preferably, a tool comprises a cutter, for instance a fine or rough diamond milling cutter, a (“twin-cut”) ball cutter, a pin cutter, a spiral or straight craniotome cutter, etc., and/or a drill, for instance a spiral drill, and/or a burnishing head and/or a swivel knife. Furthermore, the tool may also be a non-moved tool, for instance an electric scalpel, a cauter, a laser or the like. Nominal diameters of the tool may preferably range from 1.0 mm up to 6.0 mm.

According to a third aspect of the present disclosure there is proposed a medical product set having at least one first instrument handpiece according to the disclosure in combination with at least one accessory part. Preferably, the medical product set is an application-oriented assortment for an operator like a surgeon. First of all it is preferred that an instrument handpiece according to the disclosure is combined with a surgical instrument according to the disclosure. Alternatively or cumulatively, the combination with at least one accessory part of the product set comprises a plurality of different medical tools, in particular of rotatably driven and/or drivable medical tools. Thereby, this refers preferably to an assortment of the tools of different functions and types, like for instance drills, cutters and so on, and/or of a straight and/or curved shape and/or according to different sizes, as for instance pediatric, standard or short, long, and so on, and/or according to degrees of hardness and/or according to materials. Alternatively or cumulatively, the combination with at least one accessory part of the product set comprises a second instrument handpiece according to the disclosure, wherein the first instrument handpiece and the second instrument handpiece have different first diameters and/or different second diameters and/or different first lengths and/or different second lengths. Alternatively or cumulatively, the combination with at least one accessory part comprises a tool wrench for the insertion of a corresponding tool into the instrument handpiece.

Such a product set has the particular advantage that it is guaranteed by the manufacturer that the accessory parts which will be used by a user like the operator and/or a hospital staff are and may be matched such that they optimally fit together and are functionally adapted to each other. Such a product set is regarded by the user as particularly useful. Further application-technical advantages are reflected in an increased flexibility, a safe handling as well as in improved logistic hospital processes, not only in the surgical preparation but also in the Central Sterile Services Department (CSSD).

According to a fourth aspect of the present disclosure there is proposed a rinsing device, like a cleaning and disinfection unit, which is adapted for the internal flushing of an instrument handpiece according to the disclosure. A cleaning and disinfection unit (CDU), also referred to as thermodisinfector, is used for the machine-based decontamination, reprocessing or sterilization of reusable medical products like surgical instruments. Consequently, the Central Sterile Services Department (CSSD) is provided by the manufacturer with a device which is optimally adjusted to the cleaning of the instrument handpiece according to the disclosure.

According to a fifth aspect of the present disclosure, a cleaning method for the internal flushing of an instrument handpiece according to the disclosure in the flow direction from proximal to distal in a rinsing device according to the disclosure is proposed. In this manner, the cleaning efficiency and the obtainable degree of sterilization are improved.

Thereby, preferably the cleaning method according to the disclosure is further optimized hydrodynamically and is developed such that flow lines of a cleaning fluid which preferably run through the proximal roller bearing also include such flow lines which run along an outer lateral surface of the continuously formed bearing cage and/or through the at least one distal roller bearing. Thereby an incomplete or inadequate through-flow with the cleaning fluid can further be avoided.

In a first alternative preferred embodiment, a surgical instrument handpiece for a surgical instrument relating to the first aspect of the disclosure comprises:
a handle section for proximal handling by an operator, and
a shaft section which extends from the handle section in a distal longitudinal direction, wherein a tool is arranged or arrangeable by a user in a distal outlet opening of the shaft section at the distal end opposite the handle section,

wherein the shaft section has at least one narrowed distal tip section with a reduced cross-sectional area in the region of the distal outlet opening; and

wherein the shaft section comprises an inner roller bearing arrangement for a rotatable mounting of the arranged or arrangeable tool, wherein the roller bearing arrangement comprises at least one distal roller bearing and at least one proximal roller bearing, and wherein the at least one distal roller bearing and the at least one proximal roller bearing are arranged in a spaced apart manner by means of a bearing cage continuously formed between them.

Preferably, in said surgical instrument handpiece according to the first alternative preferred embodiment relating to the first aspect of the disclosure, a first diameter of the narrowed distal tip section is one or both of the following:
smaller in comparison with a second diameter of a non-narrowed region of the shaft section by a diameter relation factor of a maximum of 95 percent; and
between 3.5 and 5.3 millimeter.

Preferably, in said surgical instrument handpiece according to the first alternative preferred embodiment relating to the first aspect of the disclosure, a first length of the distal tip section is one or both of the following:
of a length proportion in relation to an entire second length of the shaft section of at least 5 percent; and
between 5 and 40 millimeter.

Preferably, in said surgical instrument handpiece according to the first alternative preferred embodiment relating to the first aspect of the disclosure, a transition region designed as a shoulder from the narrowed distal tip section to the non-narrowed region of the shaft section is one or more of being formed: in a rounded shape, in a gradually tapering off shape, and in a chamfered shape.

Preferably, in said surgical instrument handpiece according to the first alternative preferred embodiment relating to the first aspect of the disclosure, the surgical instrument handpiece is adapted to be inserted into a rinsing device in such a manner that during an internal flushing of the instrument handpiece with a cleaning fluid in the flow direction from proximal to distal the rinsing pressure being present at the distal outlet opening is one or both of the following:
larger than 10 mbar; and
in relation to a proximally applied input pressure of the cleaning fluid will remain maintained at a proportion of at least 20 percent.

Preferably, in said surgical instrument handpiece according to the first alternative preferred embodiment relating to the first aspect of the disclosure, the surgical instrument handpiece is adapted to be inserted into a rinsing device in such a manner that during an internal flushing of the instrument handpiece with a cleaning fluid in the flow direction from proximal to distal the rinsing pressure being present at the distal outlet opening is larger than 600 mbar.

Preferably, in said surgical instrument handpiece according to the first alternative preferred embodiment relating to the first aspect of the disclosure, the bearing cage is completely closed or is permeable to a fluid in a small area-related hole volume fraction of less than 40 percent.

Preferably, in said surgical instrument handpiece according to the first alternative preferred embodiment relating to the first aspect of the disclosure, one or both of the at least one distal roller bearing and the at least one proximal roller bearing comprise(s) non-spherical rolling elements.

Preferably, in said surgical instrument handpiece according to the first alternative preferred embodiment relating to the first aspect of the disclosure, one or both of the at least one distal roller bearing and the at least one proximal roller bearing comprise(s) ceramic rolling elements.

Preferably, in said surgical instrument handpiece according to the first alternative preferred embodiment relating to the first aspect of the disclosure, a bearing cage ring gap cross-sectional area of a ring gap arranged in the narrowed distal tip section, wherein said ring gap is formed at an outer lateral surface of the continuously formed bearing cage between an inner surface section diameter of the inner surface section as a ring gap outer diameter and a bearing cage outer diameter as a ring gap inner diameter, is one or both of the following:
less than or equal to 3.5 mm², and
less than or equal to a proximal shaft section ring gap cross-sectional area of the shaft section.

In the first alternative preferred embodiment, a surgical instrument relating to the second aspect of the disclosure comprises an instrument handpiece (according to the first alternative preferred embodiment) relating to the first aspect of the disclosure and a driven and/or drivable tool.

In the first alternative preferred embodiment, a medical product set relating to the third aspect of the disclosure has at least one first instrument handpiece (according to the first alternative preferred embodiment) relating to the first aspect of the disclosure, in combination with one or more of:
one surgical instrument (according to the first alternative preferred embodiment) relating to the second aspect of the disclosure;
a plurality of different medical tools;
a second instrument handpiece (according to the first alternative preferred embodiment) relating to the first aspect of the disclosure, wherein the first instrument handpiece and the second instrument handpiece have one or more of different first diameters, different second diameters, different first lengths, and different second lengths; and
a tool wrench for the insertion of a corresponding tool into the instrument handpiece.

In the first alternative preferred embodiment, a rinsing device relating to the fourth aspect of the disclosure is adapted for an internal flushing of the instrument handpiece (according to the first alternative preferred embodiment) relating to the first aspect of the disclosure.

In the first alternative preferred embodiment, a cleaning method relating to the fifth aspect of the disclosure is configured for the internal flushing of the instrument handpiece (according to the first alternative preferred embodiment) relating to the first aspect of the disclosure, in a flow direction from proximal to distal in the rinsing device (according to the first alternative preferred embodiment) relating to the fourth aspect of the disclosure.

Preferably, in said cleaning method according to the first alternative preferred embodiment relating to the fifth aspect of the disclosure, the cleaning method is configured for an instrument handpiece according to the first alternative preferred embodiment relating to the first aspect of the disclosure, wherein flow lines of a cleaning fluid running through the proximal roller bearing include such flow lines according to one or both of the following:
that run along the outer lateral surface of the continuously formed bearing cage; and
that run through the at least one distal roller bearing.

In a second alternative preferred embodiment, a surgical instrument handpiece for a surgical instrument relating to the first aspect of the disclosure comprises:
a handle section for proximal handling by an operator, and
a shaft section which extends from the handle section in a distal longitudinal direction, wherein a tool is arranged or arrangeable by a user in a distal outlet opening of the shaft section at the distal end opposite the handle section,

wherein the shaft section has at least one narrowed distal tip section with a reduced cross-sectional area in the region of the distal outlet opening; and

wherein the surgical instrument handpiece is adapted to be inserted into a rinsing device in such a manner that during an internal flushing of the instrument handpiece with a cleaning fluid in the flow direction from proximal to distal, the rinsing pressure being present at the distal outlet opening is one or both of the following:
larger than 10 mbar,
   further preferred larger than 90 mbar, even more preferred larger than 160 mbar and in particular larger than 500 mbar; and
in relation to a proximally applied input pressure of the cleaning fluid will remain maintained at a proportion of at least 20 percent,
   preferably of at least 50 percent, further preferred of at least 80 percent.

Thereby, the rinsing device may preferably be a cleaning and disinfection unit. Thereby the cleaning fluid may preferably be a hydrophilic or lipophilic cleaning solution.

Preferably, in said surgical instrument handpiece according to the second alternative preferred embodiment relating to the first aspect of the disclosure, a first diameter of the narrowed distal tip section is one or both of the following:
smaller in comparison with a second diameter of a non-narrowed region of the shaft section by a diameter relation factor of a maximum of 95 percent; and
between 3.5 and 5.3 millimeter.

Preferably, in said surgical instrument handpiece according to the second alternative preferred embodiment relating to the first aspect of the disclosure, a first length of the distal tip section is one or both of the following:
of a length proportion in relation to an entire second length of the shaft section of at least 5 percent; and
between 5 and 40 millimeter.

Preferably, in said surgical instrument handpiece according to the second alternative preferred embodiment relating to the first aspect of the disclosure, a transition region designed as a shoulder from the narrowed distal tip section to the non-narrowed region of the shaft section is one or more of being formed: in a rounded shape, in a gradually tapering off shape, and in a chamfered shape.

Preferably, in said surgical instrument handpiece according to the second alternative preferred embodiment relating to the first aspect of the disclosure, the shaft section comprises an inner roller bearing arrangement for a rotatable mounting of the arranged or arrangeable tool, preferably at least in part in the region of the narrowed distal tip section, wherein the roller bearing arrangement comprises at least one distal roller bearing and at least one proximal roller bearing, and wherein the at least one distal roller bearing and the at least one proximal roller bearing are arranged in a spaced apart manner by means of a bearing cage continuously formed between them.

Preferably, in said surgical instrument handpiece according to the second alternative preferred embodiment relating to the first aspect of the disclosure that comprises said bearing cage, the surgical instrument handpiece is adapted to be inserted into a rinsing device in such a manner that during an internal flushing of the instrument handpiece with a cleaning fluid in the flow direction from proximal to distal the rinsing pressure being present at the distal outlet opening is larger than 600 mbar.

Preferably, in said surgical instrument handpiece according to the second alternative preferred embodiment relating to the first aspect of the disclosure that comprises said bearing cage, the bearing cage is completely closed or is permeable to a fluid in a small area-related hole volume fraction of less than 40 percent.

Preferably, in said surgical instrument handpiece according to the second alternative preferred embodiment relating to the first aspect of the disclosure that comprises said bearing cage, one or both of the at least one distal roller bearing and the at least one proximal roller bearing comprise(s) non-spherical rolling elements.

Preferably, in said surgical instrument handpiece according to the second alternative preferred embodiment relating to the first aspect of the disclosure that comprises said bearing cage, one or both of the at least one distal roller bearing and the at least one proximal roller bearing comprise(s) ceramic rolling elements.

Preferably, in said surgical instrument handpiece according to the second alternative preferred embodiment relating to the first aspect of the disclosure that comprises said bearing cage, a bearing cage ring gap cross-sectional area of a ring gap arranged in the narrowed distal tip section, wherein said ring gap is formed at an outer lateral surface of the continuously formed bearing cage between an inner surface section diameter of the inner surface section as a ring gap outer diameter and a bearing cage outer diameter as a ring gap inner diameter, is one or both of the following:
less than or equal to 3.5 mm², and
less than or equal to a proximal shaft section ring gap cross-sectional area of the shaft section.

In the second alternative preferred embodiment, a surgical instrument relating to the second aspect of the disclosure comprises an instrument handpiece (according to the second alternative preferred embodiment) relating to the first aspect of the disclosure and a driven and/or drivable tool.

In the second alternative preferred embodiment, a medical product set relating to the third aspect of the disclosure has at least one first instrument handpiece (according to the second alternative preferred embodiment) relating to the first aspect of the disclosure, in combination with one or more of:
one surgical instrument (according to the second alternative preferred embodiment) relating to the second aspect of the disclosure;
a plurality of different medical tools;
a second instrument handpiece (according to the second alternative preferred embodiment) relating to the first aspect of the disclosure, wherein the first instrument handpiece and the second instrument handpiece have one or more of different first diameters, different second diameters, different first lengths, and different second lengths; and
a tool wrench for the insertion of a corresponding tool into the instrument handpiece.

In the second alternative preferred embodiment, a rinsing device relating to the fourth aspect of the disclosure is adapted for an internal flushing of the instrument handpiece (according to the second alternative preferred embodiment) relating to the first aspect of the disclosure.

In the second alternative preferred embodiment, a cleaning method relating to the fifth aspect of the disclosure is configured for the internal flushing of the instrument handpiece (according to the second alternative preferred embodiment) relating to the first aspect of the disclosure, in a flow direction from proximal to distal in the rinsing device (according to the second alternative preferred embodiment) relating to the fourth aspect of the disclosure.

Preferably, in said cleaning method according to the second alternative preferred embodiment relating to the fifth aspect of the disclosure, the cleaning method is configured for an instrument handpiece according to the second alternative preferred embodiment relating to the first aspect of the disclosure, wherein flow lines of a cleaning fluid running through the proximal roller bearing include such flow lines according to one or both of the following:
that run along the outer lateral surface of the continuously formed bearing cage; and
that run through the at least one distal roller bearing.

Finally it should be pointed out that the instrument handpiece according to the disclosure is not only restricted solely to the use in surgery. The disclosure is equally advantageously applicable for similar medical uses, in particular for the variety of dental as well as orthopedic situations and measures as well as of diagnostic methods or examination methods. The field of application includes human medicine as well as veterinary medicine. The concept according to the invention is directed towards any field of application of an instrument handpiece for the reception of an in particular rotatably mounted tool in which a reliable cleaning via an internal flushing after the use or after the removal of the tool is of a significant importance for a reuse, and/or in which a distal constructional design as small as possible is essential.

The scope of protection of the present disclosure is given by the claims and is not restricted by the features as described in the description or as shown in the figures.

### BRIEF DESCRIPTION OF THE DRAWING FIGURES

FIG. 1 is a slightly perspective lateral view of an instrument handpiece (without tool) in an embodiment according to the prior art;

FIG. 2 is a lateral view of the instrument handpiece (without tool) in the embodiment according to the prior art;

FIG. 3a is a distal detailed part of a lateral sectional view of the instrument handpiece (without tool) in the embodiment according to the prior art, in particular illustrating the inner roller bearing arrangement for a tool;

FIG. 3b is, corresponding to the distal detailed part of FIG. 3a, a schematic representation of hydrodynamic flow lines, in particular illustrating the inner through-flow with a cleaning fluid;

FIG. 4 is a slightly perspective lateral view of the instrument handpiece (without tool) according to the disclosure in a preferred embodiment;

FIG. 5 is a lateral view of the instrument handpiece (without tool) according to the disclosure in the preferred embodiment;

FIG. 6a is a distal detailed part of a lateral sectional view of the instrument handpiece (without tool) according to the disclosure in the preferred embodiment, in particular illustrating the inner roller bearing arrangement for a tool;

FIG. 6b is, corresponding to the distal detailed part of FIG. 6a, a schematic representation of hydrodynamic flow lines, in particular illustrating the inner through-flow through the instrument handpiece (without tool) according to the disclosure with a cleaning fluid corresponding to the preferred embodiment;

FIG. 7 is a slightly perspective lateral view of a preferred bearing cage in the form of an excerpt representation as a separate component for the inside of the instrument handpiece according to the disclosure corresponding to the preferred embodiment;

FIG. 8a is a first sectional view of the instrument handpiece (without tool), showing a region of a tool receptacle in a shaft section, wherein said region is proximal to a proximal roller bearing, in the embodiment according to the prior art;

FIG. 8b is a second sectional view of the instrument handpiece (without tool), wherein said second sectional view is distal to the first sectional view of FIG. 8a and shows the proximal roller bearing in the shaft section, in the embodiment according to the prior art;

FIG. 9 is a sectional view of the instrument handpiece according to the disclosure, showing a region in the transition to a handle section, wherein said region is proximal to the shaft section;

FIG. 10a is a first sectional view of the instrument handpiece (without tool) according to the disclosure, showing the region of the tool receptacle in the shaft section, wherein said region is proximal to the proximal roller bearing, according to the preferred embodiment; and

FIG. 10b is a second sectional view of the instrument handpiece (without tool) according to the disclosure, wherein said second sectional view is distal to the first sectional view of FIG. 10a and shows a central region of the bearing cage according to FIG. 7 in the narrowed distal tip section, according to the preferred embodiment.

### DETAILED DESCRIPTION

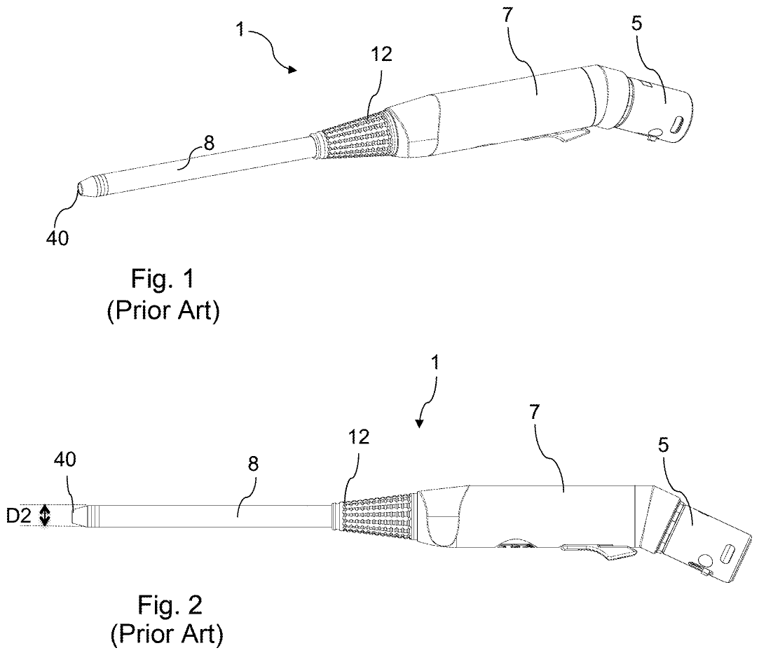

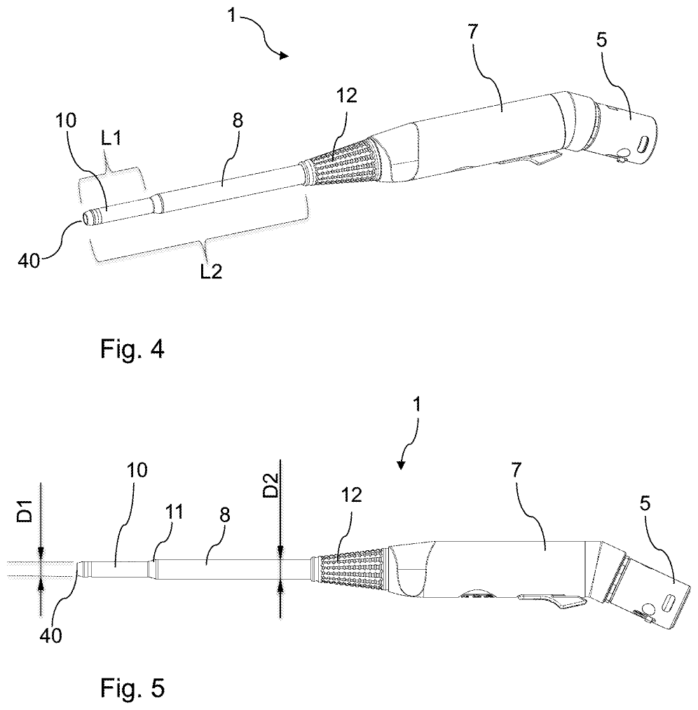

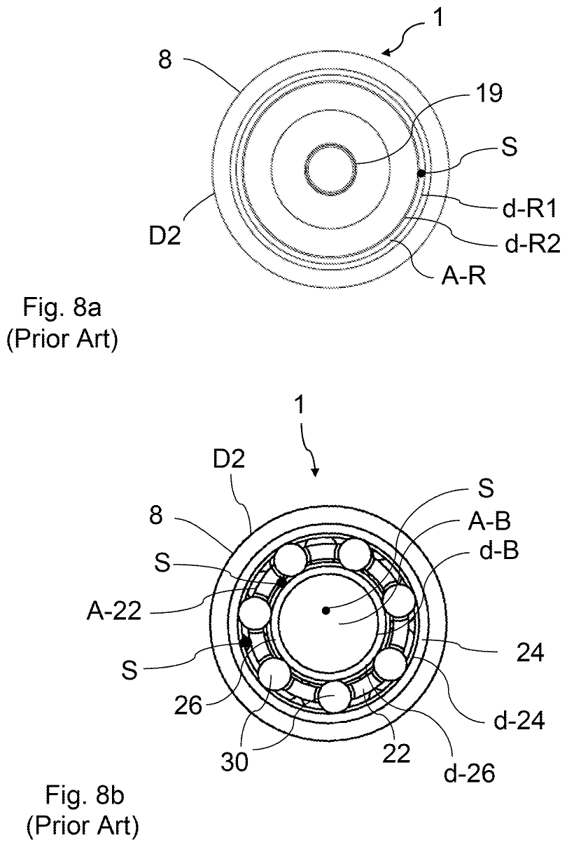

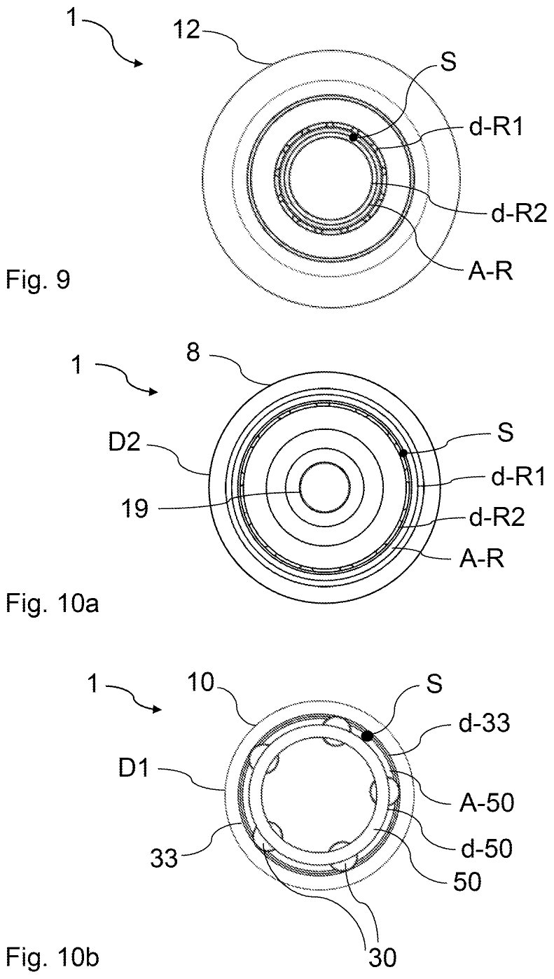

In the following, an embodiment of the present disclosure will be described on the basis of the corresponding FIGS. 4 to 6b, FIG. 7, as well as FIGS. 9 and 10, and in this respect it will be contrasted with an embodiment according to the prior art corresponding to FIGS. 1 to 3b as well as FIGS. 8a and 8b. Therefrom further details, features and advantages of the disclosure become apparent.

Insofar as the instrument handpiece according to the disclosure corresponding to the preferred embodiment according to FIGS. 4 to 6b as well as FIGS. 10a and 10b is identical to the embodiment according to the prior art corresponding to the analogous FIGS. 1 to 3b as well as according to the analogous FIGS. 8a and 8b, or insofar as a distinguishing characteristic according to the disclosure is not discussed, reference will be made to the introductory description and to the designations with regard to the prior art in order to avoid repetitions.

FIG. 1 and FIG. 2 show a slightly perspective lateral view and a lateral view of an instrument handpiece (without tool) in an embodiment according to the prior art. A surgical instrument handpiece 1 for a surgical instrument comprises an integrally formed handle section 7 which can be handled by an operator (not shown) proximally/in a manner facing away from the patient, as well as a shaft section 8 which extends from the handle section in a distal longitudinal direction/in a longitudinal direction facing the patient. Thereby, a tool (not shown), as for instance a diamond milling cutter or a spiral drill, is arrangeable by a user at the distal end opposite the handle section 7 in a cylinder bore 40 as a distal outlet opening of the shaft section 8. Thereby, the tool (not shown) typically comprises a tool head, like a drill head, a cutter head, a grinding head or a burnishing head, and a tool shaft for the insertion into the cylinder bore 40.

Furthermore, at the end proximally (facing away from the patient) the instrument handpiece 1 comprises a connection 5 by means of which it can be connected to a torque transmission train, a drive unit, an energy supply unit or the like, as known from the prior art.

Between the cylinder bore 40 at the distal end adapted for the reception of the tool and the connection 5 there is formed a handle section 7 with a surface profiling 12 (knobs, grooves, etc.) which is adjoined by a cylindrical shaft section 8 in the direction towards distal. The surface profiling 12 consists of radial and axial recesses between which protrusions are formed. Usually, the instrument handpiece 1 will be grasped by the operator at the handle section 7 and will be handled at the handle section 7 during the use thereof.

The cylindrical shaft section 8 is formed with a constant second diameter D2 (see FIG. 2).

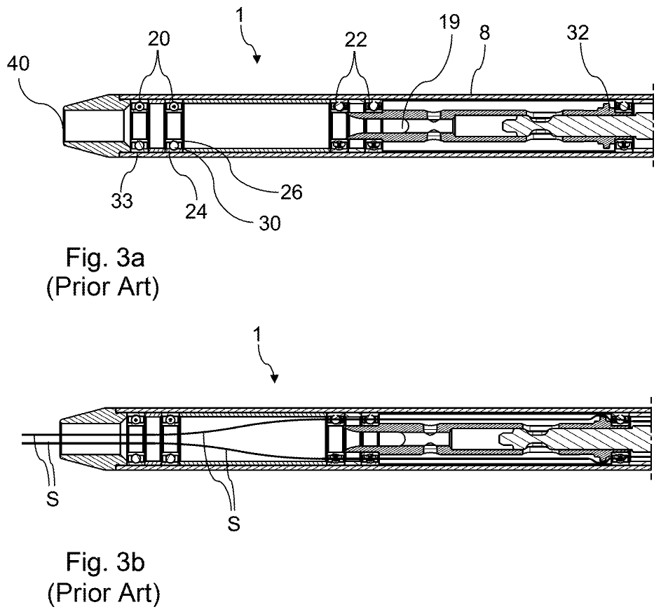

Furthermore, FIG. 3a and FIG. 3b each show the same distal detailed part of a lateral sectional view of the instrument handpiece (without tool) according to the prior art: on the one hand (FIG. 3a) without any through-flow in the sense of a normal workshop drawing, on the other hand in the fashion of a hydrodynamic schematic representation in a state with a fluid flowing therethrough (FIG. 3b). For reasons of clarity, in FIG. 3b only the flow lines S are provided with a reference numeral, which is why in the sense of the following description reference is made to the designation of the components with reference numerals in the corresponding FIG. 3a.

Said representations of FIG. 3a and FIG. 3b show in particular the inner entire roller bearing arrangement. Said inner roller bearing arrangement is provided for rotatably arranging a rotatably drivable tool (not illustrated) in the inside of the instrument handpiece 1, in particular of the shaft section 8. In this respect, the views of FIG. 3a and FIG. 3b are discontinued, as is to be suggested by the dot-dashed line (at the right edge of the illustration). The entire roller bearing arrangement comprises a distal ball bearing pair 20, formed of two distal roller bearings (on the left side of the illustration), and a proximal roller bearing pair 22 formed of two proximal roller bearings 22 (on the right side of the illustration). Thereby, all four individual ball bearings, namely the respective ones of the distal ball bearing pair 20 and of the proximal ball bearing pair 22, are all of the same construction. To this end, each individual ball bearing comprises a plurality of balls 30 as spherical rolling elements which roll between a respective inner ring 26 and a respective outer ring 24 or which roll off thereon, whereby said rings are spaced apart from each other. The respective outer ring 24 is fitted in a distal cylindrical inner surface section 33 of the shaft section 8.

Furthermore, in said sectional view of FIG. 3a and FIG. 3b there are visible an inner tool receptacle 19 for holding or anchoring the tool shaft (not shown) as well as a guide bushing 32 at the proximal end of the shaft section 8. The tool (not shown) which is inserted through the cylinder bore 40 at the distal tool end is preferably exchangeably held or coupled in the tool receptacle 19 and the guide bushing 32 of the instrument handpiece 1 and can be rotatably driven via the proximal connection 5 (see FIGS. 1 and 2).

The hydrodynamic schematic representation of FIG. 3b illustrates a state of the instrument handpiece 1 where a flow is flowing therethrough, by means of linearly drawn flow lines S. Such a state with a flow flowing therethrough occurs when the instrument handpiece 1 is inserted into a (not shown) rinsing device, like a cleaning and disinfection unit. The longitudinal flow lines S represent an internal flushing of the instrument handpiece 1 with a cleaning fluid, preferably with a hydrophilic or lipophilic cleaning solution, in the flow direction from proximal to distal. In this respect the flow lines S exit from the distal outlet opening 40 (on the left side of the illustration).

As regards the through-flow through the entire roller bearing arrangement in the direction from the proximal ball bearing pair 22 (on the right side of the illustration) up to the distal ball bearing pair 20 (on the left side of the illustration), said through-flow becomes clearly recognizable by means of the course of the flow lines S of FIG. 3b: First of all, a forced through-flow which is caused by the constructional design in the proximal direction—and, consequently, a hydrodynamically effective cleaning—of the proximal ball bearing pair 22 takes place by flowing around the corresponding balls 30. When exiting the proximal ball bearing pair 22, downstream/towards distal the flow will, however, look for a flow path substantially towards the center axis of the shaft section (in accordance with the path of least flow resistance). Finally, the most part of the cleaning fluid flows through the comparatively large cylindrical opening or bore of the paired inner rings 26, 26 of the distal ball bearing pair 20 and then exists via the distal outlet opening 40 out of the inside of the shaft section 8. In this respect there is hardly any flow through the distal ball bearing pair 20 (on the left side of the illustration) according to the prior art and, thus, the distal ball bearing pair 20 will also not be adequately cleaned (by the fluid).

The above described course of the through-flow or the flow conditions from proximal to distal through the instrument handpiece 1 in the embodiment according to the prior art will be further illustrated in detail by means of FIGS. 8a and 8b. FIG. 8a, for instance, shows a first sectional view of the conventional instrument handpiece 1 (without tool) which refers to a cross-section through the cylindrical shaft section 8 with a constant second diameter D2 (see also FIG. 2). Here, the first sectional view of FIG. 8a lies within a region of the tool receptacle 19 (see also FIG. 3a) which is arranged more proximal than the proximal roller bearing 22 (FIG. 22). Furthermore, FIG. 8b shows a second sectional view of the conventional instrument handpiece (without tool) which is sectioned distally or in the flow direction further downstream when compared to the first sectional view of FIG. 8a. Thereby, the second sectional view of FIG. 8b sections the proximal roller bearing 22 in the shaft section 8 with the second diameter 8.

Thereby, in the first and second sectional view (for the prior art: in FIGS. 8a and 8b) reference is particularly made to the hydrodynamic schematical view (for the prior art: in FIG. 3b) which illustrates the state of the instrument handpiece 1 when a fluid is flowing therethrough by means of the linearly drawn flow lines S (or an exemplary selection of the two flow lines from actually a plurality).

Thereby, the flow lines S step out of a sheet plane which is related to the representation of the sectional view(s) (for the prior art: in FIGS. 8a and 8b); that is to say (ideally) punctiformly in the direction of the viewer. Like, so to speak, an arrow head of an accordingly corresponding flow vector of a flow rate which passes through the sheet plane, a respective flow line S (represented by example/in a selection) is represented.

Thus, the respective flow line S which is punctiform in the sectional view indicates a cross-sectional area through which a flow is flowing or which is open, respectively, or a (corresponding) flow cross-sectional area. In other words, a point indicated by means of the flow line S in one of the sectional views respectively implies a (flow) cross-sectional area (which is recognizable between contour lines or body edges, or which is an individual one) which is available respectively to a flow path of the through-flow. Consequently, for or during the flushing with the cleaning fluid by means of the (not illustrated) rinsing device, preferably by means of the cleaning and disinfection unit, the (respective flow) cross-sectional area is in a fluid communication with an input connection of the instrument handpiece 1.

From the first sectional view of FIG. 8a it can be inferred that a cylindrical ring gap cross-sectional area A-R is formed between an outer cylindrical ring gap outer diameter d-R1 and an inner ring gap inner diameter d-R2 for the through-flow (with a punctiformly exiting flow line S). Hence, the ring gap cross-sectional area A-R is calculated as a (respective flow) cross-sectional area on the basis of the subtraction of the two circular areas with the ring gap outer diameter d-R1 or, respectively, with the ring gap inner diameter d-R2. The ring gap cross-sectional area A-R as is shown in FIG. 8a may for instance amount to 2.4 mm².

The second sectional view of FIG. 8b shows the flow situation downstream/distally. Here, in the proximal roller bearing 22 the through-flow flows through two flow spaces or respective (flow) cross-sectional areas which are separated by the inner ring 26. Thereby, these taken together result in an entire roller bearing flow cross-section (for instance 8.5 mm²), as will be explained in the following: The proximal roller bearing 22 is represented with seven balls 30 as the rolling elements which roll in a rolling off manner between the inner ring 26 with the inner ring diameter d-26 and the outer ring 24 fitted in the shaft section 8 and having the outer ring diameter d-26. A part of the through-flow flows (with a punctiformly exiting flow line S) centrally through a cylindrical bore inner space of the inner ring 26 with a bore cross-sectional area A-B (for instance 4.5 mm²) according to a bore diameter d-B (for instance 2.4 mm).

In addition, the other part of the through-flow flows through a roller bearing inner space of the proximal roller bearing 22 with a (free) roller bearing cross-sectional area A-22 (for instance 4.0 mm²), wherein said roller bearing inner space is formed between the inner ring 26 and the outer ring 24 and is free from the (seven) balls 30, with two punctiformly exiting flow lines S.

According to the hydrodynamic law of the path according to the least flow resistance and/or according to the flow condition known as wall adhesion condition, in particular in the case of a pipe flow, and/or according to the through-flow through a bed (“Pre-Darcy”), there results hydrodynamically a distribution of the flow rate (in the longitudinal direction of the instrument handpiece 1) with a maximum within the bore cross-sectional area A-B. In contrast thereto, only a small portion of the other part of the through-flow or a portion relatively small thereof when compared to the one part will pass through the roller bearing cross-sectional area A-22.

In other words, the structure of the conventional instrument handpiece 1 causes in a disadvantageous manner that, when there is a through-flow with a cleaning fluid flowing therethrough, especially the roller bearing (or the here representatively hydrodynamically discussed proximal roller bearing 22) will only experience a low or weak or slow through-flow. In the prior art that circumstance is considered to be particularly disadvantageous in that the flow cross-section is not only not tapered from proximal (the ring gap cross-sectional area A-R, for instance 2.4 mm²), but even considerably enlarges towards distal (entire roller bearing flow cross-section for instance 8.5 mm²). As a consequence, the risk exists that a mechanical cleaning effect by means of the cleaning fluid will not ensue in an adequate manner. In particular, the mechanical cleaning effect is determined according to the kinetic energy of the cleaning fluid as a hydrodynamic parameter into which, in turn, the flow rate squared is incorporated.

The above explained disadvantage of the reduced through-flow in the roller bearing as a result thereof and the, thus, reduced cleaning effect is all the greater in terms of the technical aim of hygienics, in particular in terms of a completely reliable sterilization, inasmuch as exactly the large surfaces of the roller bearings offer particularly much surface area for the adhesion of contaminations like germs, biofilms and the like.

Thus, on the basis of the first and second sectional view for the conventional instrument handpiece in the FIGS. 8a and 8b the disadvantage of the prior art as described already above with the aid of FIG. 3b becomes especially evident, namely as regards the inadequate (fluid) cleaning effect of the through-flow visualized by means of the flow lines S.

According to the present disclosure, these disadvantages are remedied. FIGS. 4 to 7 show different views according to an embodiment of an instrument handpiece 1 according to the disclosure. For instance, FIG. 4 and FIG. 5 show [in analogy to FIG. 1 and FIG. 2 for the prior art] a slightly perspective lateral view and a lateral view of the instrument handpiece (without tool) according to the disclosure in a preferred embodiment.

Unlike in the prior art, the shaft section 8 comprises a narrowed distal tip section 10 in the region of the distal outlet opening 40 (on the left side in FIGS. 4 to 6b). The distal tip section 10 is narrowed/reduced from a second diameter D2 of the shaft section 8 down to a smaller first diameter D1 (see FIG. 5). In other words, the preferred embodiment as shown in FIGS. 4 to 7 of an instrument handpiece according to the disclosure differs from the conventional instrument handpiece according to the prior art as shown in FIGS. 1 to 3b in that the longitudinal shaft section 8 with a second diameter D2 in its distal region of the cylinder bore 40 as a distal outlet opening has a narrowed distal tip section 10.

As is indicated in FIG. 4 by means of curly brackets, the first length L1 of the distal tip section 10 occupies a distal partial section of the (entire) second length L2 of the (entire) shaft section 8. Between the distal tip section 10 with the first diameter D1 and the shaft section 8 with the second diameter D2 there is circumferentially formed a shoulder or a beveled step as a transition region 11 (see FIG. 5) in a gradually tapering off or chamfered manner.

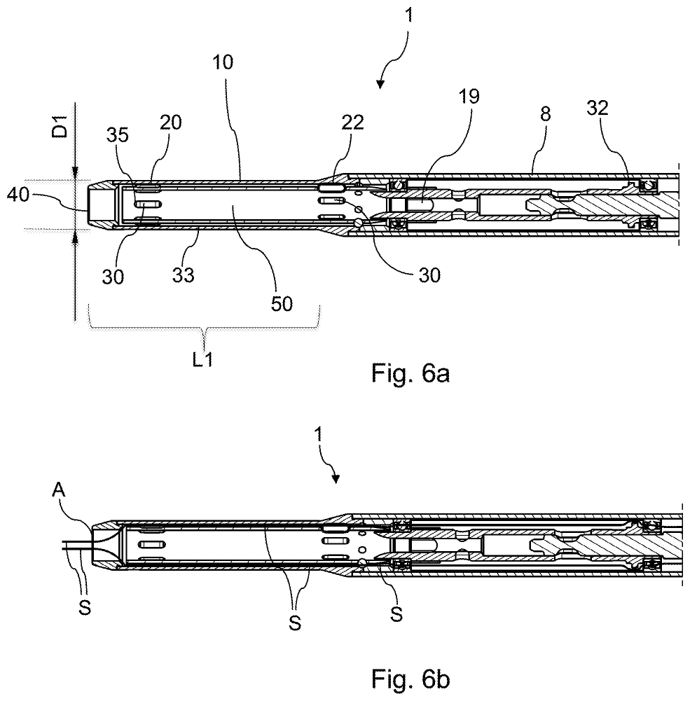

Furthermore, FIG. 6a and FIG. 6b show [by analogy to FIG. 3a and FIG. 3b for the prior art] respectively the same distal detailed part of a lateral sectional view of the instrument handpiece (without tool) according to the disclosure in the preferred embodiment: on the one hand (FIG. 6a) without any through-flow in the sense of a workshop drawing; on the other hand in a state with a fluid flowing therethrough (FIG. 6b). For reasons of clarity, in FIG. 6b only the flow lines S as well as the outlet cross-sectional area A for the flow are provided with a reference numeral, which is why in the sense of the following description reference is made to the designation of the components with reference numerals in the corresponding FIG. 6a.

Said representations of FIG. 6a and FIG. 6b show in particular the inner entire roller bearing arrangement. Said inner roller bearing arrangement is provided for rotatably arranging a rotatably drivable tool (not illustrated) in the inside of the instrument handpiece 1, in particular in the distal tip section 10 of the shaft section 8. The views of FIG. 6a and FIG. 6b (as already the views of FIG. 3a and FIG. 3b) are discontinued, as is suggested by the dot-dashed line (at the right edge of the illustration). The entire roller bearing arrangement comprises a distal needle bearing (or cylindrical roller bearing) 20 (on the left side in the illustration) and a proximal needle bearing (or cylindrical roller bearing) 22 of the same construction (on the right side of the illustration). To this end, the distal needle bearing 20 and the proximal needle bearing 22 each comprise a plurality of preferably ceramic needles 30 as elongated or longitudinal unspherical rolling elements, distributed in uniform angular segments on a respective periphery of a circle. The respective needles 30 are set or arranged in a plurality of corresponding longitudinal grooves 35 in the bearing cage 50 in a manner rotatably movable around their longitudinal central axis. The distal needle bearing 20 and the proximal needle bearing 22 are arranged in a manner spaced apart from each other in the longitudinal direction of the shaft section 8 by means of a cylindrical bearing cage 50 being inserted in the cylindrical inner bore of the distal shaft section 10. In this respect, the respective needles 30 of the distal needle bearing 20 and of the proximal needle bearing 22 roll on a or roll off internally on a cylindrical inner surface section 33 of the distal tip section 10 or of the shaft section 8. Comparable to the prior art, at the proximal end of the shaft section 8 an inner tool receptacle 19 for holding or anchoring the tool shaft (not shown) as well as a guide bushing 32 are recognizable.

FIG. 6b shows, as already FIG. 3b for the prior art, a schematic representation of hydrodynamic flow lines S. Thus, as far as no differences to the prior art are affected, in order to avoid repetitions reference is made to the explanations regarding FIG. 3b. In contrast to FIG. 3b, FIG. 6b illustrates a state with a fluid through-flow according to the disclosure during an inner through-flow with a cleaning fluid through the instrument handpiece (without tool) according to the disclosure corresponding to the preferred embodiment. Such a state with a fluid through-flow according to the disclosure may in a preferable manner be effected or implemented by the fact that the instrument handpiece 1 according to the disclosure is inserted into a (not shown) rinsing device according to the disclosure, as for instance into a cleaning and disinfection unit. The longitudinal flow lines S represent an internal flushing of the instrument handpiece 1 with a cleaning fluid, preferably with a hydrophilic or lipophilic cleaning solution, in the flow direction from proximal to distal (from the right side to the left side in the illustration). In this respect, the flow lines S exit from the distal outlet opening 40 (on the left side of the illustration) of the narrowed distal tip section 10 with a correspondingly reduced outlet cross-sectional area A of the flow.

By means of the course of the flow lines S of FIG. 6b the through-flow according to the disclosure also through the distal needle bearing 20 as a distal roller bearing is clearly recognizable. Almost along the entire length of the shaft section 8, in particular on the first length L1 of the distal tip section, the flow lines run along the inner surface section 33 of the distal tip section 10 or of the shaft section 8. In particular, only after passing also through the distal needle bearing 20 or only shortly before the flow exit from the distal outlet opening 40 with a reduced outlet cross-sectional area A the flow takes a flow path downstream substantially towards the center axis of the shaft section. Thus, according to the disclosure a hydrodynamically effective (fluid) cleaning also of the distal needle bearing 20 as a distal roller bearing takes place.

This is because, due to the narrowed distal tip section 10 according to the disclosure, for the improvement of the cleaning effect the flow velocity according to the reduced outlet cross-sectional area A and the pressure in the distal tip section 10 of the instrument handpiece 1 are increased. This is caused by the change in diameter of the distal tip section 10 from the outer diameter of 5.6 mm as an exemplary second diameter D2 to the outer diameter of 4.4 mm as an exemplary first diameter D1 in the region of the first 20 mm as an exemplary first length L1.

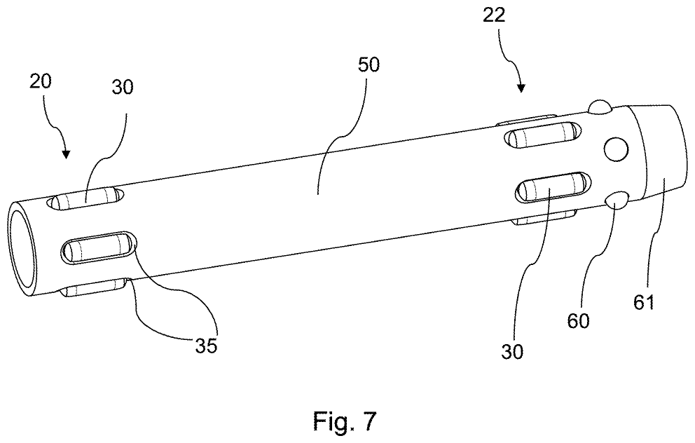

Moreover, the special constructional design of the bearing cage 50 as a continuous pipe provides for a lesser soiling during use and at the same time for an optimized cleaning by the targeted guidance of the cleaning fluid. A small portion of the cleaning fluid still gets through the tool opening 40 as a distal outlet opening of the instrument handpiece 1 and here also provides for an optimal cleaning, as no obstructing parts block the flow of the cleaning fluid. FIGS. 6a and 6b illustrate the constructive details with regard to the installation or the mounting of the bearing cage 50 which can be seen in greater detail as an individual component in FIG. 7.

The technical effect on the course of the flow lines S due to the constructional design as well as due to the arrangement of the particularly preferred embodiment with a bearing cage 50 is in particular again comprehensible by means of FIG. 6b. It can be recognized that the bearing cage 50 even causes a forced through-flow through the distal needle bearing 20, that is to say said needle bearing 20 will be passed in any case and, thus, cleaned (by the fluid). In particular by the installation of such a bearing cage 50, in addition a large degree of independence from a proximally applied input pressure of the cleaning fluid is achieved in a preferable manner, which further promotes the stability of a cleaning method according to the disclosure.

FIG. 7 shows in the form of an enlarged excerpt representation by reference to FIG. 6a and FIG. 6b, a slightly perspective lateral view of a bearing cage 50, in a manner as it may preferably be provided as a separate component of a roller bearing arrangement for the tool in the inside of the instrument handpiece 1 according to the disclosure. From said representation of FIG. 7 it can be clearly inferred that the bearing cage 50 in the form of a cylindrical pipe arranges a distal roller bearing 20 (on the left side of the illustration) and a proximal roller bearing 22 (on the right side of the illustration) in a spaced apart manner. Thereby, the distal roller bearing 20 and the proximal roller bearing 22 are implemented by means of respectively five needles 30 as unspherical rolling elements uniformly distributed at the circumference of the bearing cage 50. The needles 30 in turn are arranged in corresponding longitudinal grooves 35 of the bearing cage 50 in a manner being rotatable/rolling-off around their longitudinal axis. Furthermore, at the proximal end of the bearing cage 50 there can be recognized a plurality of spherical guide elements 60 as well as a slipping-off surface 61.

FIG. 9 shows a sectional view of the instrument handpiece 1 according to the disclosure, which depicts a region in the transition 12 to the handle section 7 (according to FIG. 4), wherein said region is proximal to the shaft section 8. It can be derived that a (cylindrical) ring gap cross-sectional area A-R (for instance approximately 3.6 mm²) which belongs to the cross-section of FIG. 9 is formed between a corresponding outer cylindrical ring gap outer diameter d-R1 (for instance approximately 3.2 mm) and a corresponding inner ring gap inner diameter d-R2 (for instance approximately 2.4 mm) for the through-flow (with a punctiformly exiting flow line S).

Furthermore, FIG. 10a and FIG. 10b show [by analogy to FIG. 8a and FIG. 8b for the prior art] a first and a second lateral view of the instrument handpiece 1 (without tool) according to the disclosure in the preferred embodiment according to FIGS. 4 to 7: First of all FIG. 10a shows a first sectional view in a region of the tool receptacle 19 in the shaft section 8 with the second diameter D2, wherein said region is proximal to the proximal roller bearing 22 (according to FIG. 6a).

And furthermore FIG. 10b shows a second sectional view of a central region of the bearing cage 50 (according to FIGS. 6a and 7) in the narrowed distal tip section 10 according to the disclosure with the first diameter D1, wherein said second sectional view is distal to the first sectional view of FIG. 10a.

By analogy to the above discussion for the prior art (on the basis of FIGS. 8a and 8b referring thereto), in the first and second sectional view of FIGS. 10a and 10b reference is made in particular to the hydrodynamic schematic representation in FIG. 6b which illustrates the state of the instrument handpiece 1 when a flow is flowing therethrough by means of the linearly drawn flow lines S (or an exemplary selection of the two flow lines actually from a plurality).

From FIG. 10a it can be inferred that a (cylindrical) ring gap cross-sectional area A-R (for instance approximately 3.6 mm²) belonging to the cross-section of the shaft section 8 in FIG. 10a is formed between a corresponding outer cylindrical ring gap outer diameter d-R1 (for instance approximately 4.5 mm) and a corresponding inner ring gap inner diameter d-R2 (for instance approximately 4.0 mm), for the through-flow (with a punctiformly exiting flow line S).

Accordingly, it may be particularly preferred that the ring gap cross-sectional area A-R belonging to the cross-section of the shaft section 8 in FIG. 10a approximately corresponds to the (cylindrical) ring gap cross-sectional area A-R belonging to the cross-section of FIG. 9 (area relation to each other of 90% to 110%, further preferred of 98% to 102%, in particular approximately 100%). This results in an advantageous uniformity of the course of the rinsing pressure or of the flow rate or of the kinetic energy in the instrument handpiece 1 from proximal to distal.

From FIG. 10b which concerns a distal cross-section according to the disclosure there can be inferred under reference to FIG. 6a and FIG. 6b that the narrowed distal tip section 10 with the first diameter D1 comprises the cylindrical (in the longitudinal direction of the instrument handpiece 1) continuous bearing cage 50 in a manner being internally spaced apart. For the through-flow (with a punctiformly exiting flow line S) there is formed a corresponding ring gap around the bearing cage 50. On the other hand, at least the proximal end, preferably also the distal end, of the bearing cage 50 is/are closed so that the central cylinder volume of the bearing cage 50 cannot be/will not be exposed to a through-flow.

Behind the cross-section of the distal tip section 10 as shown in FIG. 10b through a (in longitudinal direction) central region of the bearing cage 50 in the direction of view there can be recognized the five needles 30 as unspherical rolling elements (see FIG. 7) and being uniformly distributed at the circumference of the bearing cage 50.

Thereby, the corresponding ring gap within the inner surface section 33 is formed at an outer lateral surface of the continuously formed bearing cage 50 between an inner surface section diameter d-33 (for instance approximately 3.8 mm) as a ring gap outer diameter and a bearing cage outer diameter d-50 (for instance approximately 3.3 mm) as a ring gap inner diameter. Here, the ring gap arranged around the bearing cage 50 has a bearing cage ring gap cross-sectional area A-50 (for instance approximately 2.8 mm²).

Preferably, the bearing cage ring gap cross-sectional area A-50 may be less than or equal to 3.5 mm², further preferred less than or equal to approximately 3 mm² and particularly preferred less than or equal to 2.8 mm². Alternatively or cumulatively, the bearing cage ring gap cross-sectional area A-50 may be less than or equal to a flow cross-sectional area through which a free through-flow is possible in a region of the instrument handpiece 1 being proximal thereto, as is for instance shown in FIGS. 10a and/or 9. Further preferred, the bearing cage ring gap cross-sectional area A-50 may be less than or equal to a proximal shaft section ring gap cross-sectional area A-R (see FIG. 9).

In particular, according to the disclosure the rinsing pressure being applied or present at the distal outlet opening (with the reference numeral 40, see FIGS. 4 to 6a) or in the bearing cage ring gap cross-sectional area A-50 in the flow direction from proximal to distal can be positively increased. As a result thereof, the instrument handpiece according to the disclosure offers a remedy for a situation being technically problematic in the prior art which can occur in form of a possibly too low respective rinsing pressure in particular in a situation with a high hospital occupancy rate. As an example, the entire proximal input pressure of the cleaning fluid, which input pressure is supplied in a cleaning and disinfection unit (not shown) (for instance of the type “MIELE G 7825” construction 80) as a rinsing device from an external source to a number of several connected instrument handpieces to be cleaned, may amount to a maximum of approximately 1600 mbar in the sense of a proximal absolute pressure. In the sense of a pressure difference/an excess pressure/a pressure delta with respect to the atmospheric ambient pressure of for instance approximately 950 mbar, this results in a maximum (proximal) rinsing pressure for an individual connected instrument handpiece 1 of approximately 650 mbar. When there are several instrument handpieces present or connected, then said maximum (proximal) rinsing pressure is, however, subdivided into said several handpieces and is, thus, reduced to a respective rinsing pressure. The exemplary cleaning and disinfection unit provides a maximum of 22 connections (‘Luer-lock: type Miele”, connection inner diameter 3 mm; thus a flow cross-section: A=approximately 7 mm²). In the case of 11 connected instrument handpieces, i.e. the half, a reduction of the respective (proximal) rinsing pressure to approximately 500 mbar could be noticed proximally. In the case of 22 (of 22) connected instrument handpieces there could be observed that the respective (proximal) rinsing pressure was proximally reduced or decreased even further to approximately 315 mbar. Thus, the respective (proximal) rinsing pressure is available at the entrance of the instrument handpiece (proximal handle section flow cross-section of for instance Ø1.7 mm, A=2.27 mm²).

The (proximal) rinsing pressure applied proximally of the exemplary cleaning and disinfection unit is reduced by further taking into account various flow resistances, like the pipe friction along the inner through-flow of the instrument handpiece, from proximal to distal furthermore to a distally present (distal) rinsing pressure.

The present disclosure guarantees that also in case of a low respective rinsing pressure a reliable and adequate fluid cleaning will take place. The narrowed distal tip section 10 effects that also still in the distal region an effective rinsing pressure is present. In particular the particularly preferred embodiment with the continuously formed bearing cage 50 enables a powerful forced through-flow in the surrounding bearing cage ring gap cross-sectional area A-50. Hence, despite flow pressure losses there takes place an effective fluid cleaning even downstream/distal of the non-narrowed shaft section 8, namely in the distal tip section 10. Said fluid cleaning guarantees a quasi undiminished mechanical cleaning performance in the distal roller bearing 20 and in the proximal roller bearing 22. Insofar even the proximal roller bearing 22 receives the entire rinsing flow of the cleaning fluid.

1.-16. (canceled)

17. A surgical instrument handpiece for a surgical instrument, the surgical instrument handpiece comprising:
a handle section for proximal handling by an operator; and
a shaft section which extends from the handle section in a distal longitudinal direction, wherein a tool is arranged or arrangeable by a user in a distal outlet opening of the shaft section at a distal end opposite the handle section,
wherein the shaft section has at least one narrowed distal tip section with a reduced cross-sectional area in a region of the distal outlet opening; and
wherein the shaft section comprises an inner roller bearing arrangement for a rotatable mounting of the tool, wherein the inner roller bearing arrangement comprises at least one distal roller bearing and at least one proximal roller bearing, and wherein the at least one distal roller bearing and the at least one proximal roller bearing are arranged in a spaced apart manner by a bearing cage continuously formed between them.

18. The surgical instrument handpiece according to claim 17, wherein a first diameter of the at least one narrowed distal tip section is at least one of:
smaller in comparison with a second diameter of a non-narrowed region of the shaft section by a diameter relation factor of a maximum of 95 percent; and
between 3.5 and 5.3 millimeters.

19. The surgical instrument handpiece according to claim 17, wherein a first length of the at least one narrowed distal tip section is at least one of:
at least 5 percent of a length proportion in relation to an entire second length of the shaft section; and
between 5 and 40 millimeters.

20. The surgical instrument handpiece according to claim 17, wherein a transition region designed as a shoulder from the at least one narrowed distal tip section to the non-narrowed region of the shaft section is one or more of being formed in a rounded shape, in a gradually tapering off shape, and in a chamfered shape.

21. The surgical instrument handpiece according to claim 17, wherein the surgical instrument handpiece is adapted to be inserted into a rinsing device in such a manner that, during an internal flushing of the instrument handpiece with a cleaning fluid in a flow direction from proximal to distal, a rinsing pressure present at the distal outlet opening is at least one of:
larger than 10 mbar; and
is maintained at at least 20 percent of a proximally applied input pressure of the cleaning fluid.

22. The surgical instrument handpiece according to claim 17, wherein the surgical instrument handpiece is adapted to be inserted into a rinsing device in such a manner that, during an internal flushing of the instrument handpiece with a cleaning fluid in a flow direction from proximal to distal, a rinsing pressure present at the distal outlet opening is larger than 600 mbar.

23. The surgical instrument handpiece according to claim 17, wherein the bearing cage is completely closed or is permeable to a fluid in a small area-related hole volume fraction of less than 40 percent.

24. The surgical instrument handpiece according to claim 17, wherein one or both of the at least one distal roller bearing and the at least one proximal roller bearing comprise one or both of non-spherical rolling elements and ceramic rolling elements.

25. The surgical instrument handpiece according to claim 17, further comprising a ring gap having a bearing cage ring gap cross-sectional area, the ring gap being arranged in the at least one narrowed distal tip section, the ring gap being formed at an outer lateral surface of the bearing cage between an inner surface section diameter of an inner surface section as a ring gap outer diameter and a bearing cage outer diameter as a ring gap inner diameter, the bearing cage ring gap cross-sectional area being at least one of:
less than or equal to 3.5 mm², and
less than or equal to a proximal shaft section ring gap cross-sectional area of the shaft section.

26. A surgical instrument handpiece for a surgical instrument, the surgical instrument handpiece comprising:
a handle section for proximal handling by an operator; and
a shaft section which extends from the handle section in a distal longitudinal direction, wherein a tool is arranged or arrangeable by a user in a distal outlet opening of the shaft section at a distal end opposite the handle section,
wherein the shaft section has at least one narrowed distal tip section with a reduced cross-sectional area in a region of the distal outlet opening; and
wherein the surgical instrument handpiece is adapted to be inserted into a rinsing device in such a manner that, during an internal flushing of the instrument handpiece with a cleaning fluid in a flow direction from proximal to distal, a rinsing pressure present at the distal outlet opening is at least one of:
larger than 10 mbar; and
is maintained at at least 20 percent of a proximally applied input pressure of the cleaning fluid.

27. The surgical instrument handpiece according to claim 26, wherein a first diameter of the at least one narrowed distal tip section is at least one of:
smaller in comparison with a second diameter of a non-narrowed region of the shaft section by a diameter relation factor of a maximum of 95 percent; and
between 3.5 and 5.3 millimeters.

28. The surgical instrument handpiece according to claim 26, wherein a first length of the at least one narrowed distal tip section is at least one of:
at least 5 percent of a length proportion in relation to an entire second length of the shaft section of; and
between 5 and 40 millimeters.

29. The surgical instrument handpiece according to claim 26, wherein a transition region designed as a shoulder from the at least one narrowed distal tip section to the non-narrowed region of the shaft section is at least one of:
formed in a rounded shape;
formed in a gradually tapering off shape; and
formed in a chamfered shape.

30. The surgical instrument handpiece according to claim 26, wherein the shaft section comprises an inner roller bearing arrangement for a rotatable mounting of the tool, wherein the roller bearing arrangement comprises at least one distal roller bearing and at least one proximal roller bearing, and wherein the at least one distal roller bearing and the at least one proximal roller bearing are arranged in a spaced apart manner by a bearing cage continuously formed between them.

31. The surgical instrument handpiece according to claim 30, wherein the surgical instrument handpiece is adapted to be inserted into a rinsing device in such a manner that, during an internal flushing of the instrument handpiece with a cleaning fluid in a flow direction from proximal to distal, the rinsing pressure present at the distal outlet opening is larger than 600 mbar.

32. The surgical instrument handpiece according to claim 30, wherein the bearing cage is completely closed or is permeable to a fluid in a small area-related hole volume fraction of less than 40 percent.

33. The surgical instrument handpiece according to claim 30, wherein one or both of the at least one distal roller bearing and the at least one proximal roller bearing comprise one or both of non-spherical rolling elements and ceramic rolling elements.

34. The surgical instrument handpiece according to claim 30, further comprising a ring gap having a bearing cage ring gap cross-sectional area, the ring gap being arranged in the at least one narrowed distal tip section, the ring gap being formed at an outer lateral surface of the bearing cage between an inner surface section diameter of an inner surface section as a ring gap outer diameter and a bearing cage outer diameter as a ring gap inner diameter, the bearing cage ring gap cross-sectional area being at least one of:
less than or equal to 3.5 mm², and
less than or equal to a proximal shaft section ring gap cross-sectional area of the shaft section.

35. A rinsing device configured for an internal flushing of an instrument handpiece according to claim 17.
